(19) United States
(12) Patent Application Publication
(10) Pub. No.: US 2015/0148262 A1
(43) Pub. Date: May 28, 2015

(54) MICROPARTICLE ASSEMBLY

(71) Applicant: NATIONAL UNIVERSITY OF SINGAPORE, Singapore (SG)

(72) Inventors: Dieter Trau, Singapore (SG); Martin James Lear, Singapore (SG); Christopher Ochs, Singapore (SG)

(73) Assignee: NATIONAL UNIVERSITY OF SINGAPORE, Singapore (SG)

(21) Appl. No.: 14/402,346

(22) PCT Filed: May 22, 2013
(86) PCT No.: PCT/SG2013/000210

§ 371 Date: Nov. 20, 2014

(30) Foreign Application Priority Data
May 22, 2012 (GB) 1209034.6

(51) Int. Cl. G01N 21/64; G01N 33/545; G01N 33/58

(52) U.S. Cl. CPC G01N 21/6428; G01N 33/582; G01N 33/545; G01N 2021/6432
USPC 506/20; 506/41; 428/402

(57) ABSTRACT

A bead construct includes a bead, a fluorophore molecule, first and second linker molecules and a ligand molecule. The fluorophore molecule comprises at least two subunits connected to each other by at least one internal linker that provides conformational freedom to the at least two subunits. An optical signal emitted by the fluorophore molecule changes when reducing the conformational freedom of the at least two subunits to each other. The first linker molecule is connected to the bead and to one of the subunits of the flurophore molecule. The second linker molecule is connected to another one of the subunits of the fluorophore molecule. The ligand molecule is connected to the second linker molecule.

### CROSS-REFERENCE TO PRIOR APPLICATIONS

This application is a U.S. National Phase Application under 35 U.S.C. §371 of International Application No. PCT/SG2013/000210, filed on May 22, 2013, and claims benefit to British Patent Application No. GB 1209034.6, filed on May 22, 2012. The International Application was published in English on Nov. 28, 2013 as WO 2013/176625 under PCT Article 21(2).

### FIELD

The present invention generally relates to a microparticle assembly. The present invention also relates to a microarray comprising a plurality of microparticle assemblies and a method of manufacturing thereof. The present invention also relates to a method for identifying the presence of a target analyte in a sample and a kit for the same.

### BACKGROUND

DNA and Protein Microarrays are important tools for diagnostic applications in research and clinical settings. Microarrays carrying small molecule binding partners are not yet commercially available and still in the research stage. Microarrays with small molecule binding partners are interesting as potential research tools in the future.

Bead microarrays carry microbeads which, in turn, are conjugated to biomolecules. Bead microarrays have significant advantages such as consistency, flexibility and fast kinetics. Bead microarrays are flexible because the surface chemistry of the beads can be tailored to suit the biomolecule to be conjugated (via linkers) on the bead. Potentially, any type of microarray such as drug-, metabolite-, lipid- or carbohydrate-microarrays could be manufactured by using beads. In state-of-the-art bead microarrays, the binding molecules (typically DNA types, oligonucleotides or proteins) are directly linked to the microbead with a linker. Typical linker chemistry may use biotin-streptavidin or N-hydroxysuccinimide activated functional groups to link the biomolecule to the microbead. Further, bead microarrays ensure consistent results since the dimensions of the beads can be made uniform.

Currently, bead arrays come in the form of “liquid arrays” or “planar arrays”. In liquid arrays, beads remain in suspension while in planar arrays, beads are deposited onto a planar substrate material. In both approaches, typical DNA, protein, or antibody arrays are used today.

Both approaches, however, suffer from the need to optically label the analyte molecule that binds to the bead microarray. In DNA bead arrays, the DNA is labeled with a fluorophore or quantum dot, or other optical signatures. In antibody bead microarrays, typically a sandwich antibody assay is used with a fluorescently labeled detector antibody. The detector antibody can directly carry the optical label or a secondary optical labeled antibody is used. All such systems require the addition of one or more reagents and add complexity to the assay. Another Major drawback is the need for additional incubation steps, which increase the time required for conducting the assay.

### SUMMARY

In an embodiment, the present invention provides a bead construct including a bead, a fluorophore molecule, first and second linker molecules and a ligand molecule. The fluorophore molecule comprises at least two subunits connected to each other by at least one internal linker that provides conformational freedom to the at least two subunits. An optical signal emitted by the fluorophore molecule changes when reducing the conformational freedom of the at least two subunits to each other. The first linker molecule is connected to the bead and to one of the subunits of the flurophore molecule. The second linker molecule is connected to another one of the subunits of the fluorophore molecule. The ligand molecule is connected to the second linker molecule.

### BRIEF DESCRIPTION OF THE DRAWINGS

The present invention will be described in even greater detail below based on the exemplary figures. The invention is not limited to the exemplary embodiments. All features described and/or illustrated herein can be used alone or combined in different combinations in embodiments of the invention. The features and advantages of various embodiments of the present invention will become apparent by reading the following detailed description with reference to the attached drawings which illustrate the following:

FIGS. 1A to 1C are schematic diagrams showing possible embodiments of the microbead construct of the present invention;

FIG. 2 is a schematic diagram illustrating one of the design principles of an embodiment of the present invention in relation to the distance between two flurophores of a microbead construct of an embodiment of the present invention;

FIGS. 3A to 3E are schematic diagrams illustrating various exemplary bead bioassays involving the use of a microbead construct of an embodiment of the present invention;

FIG. 4A is a graph showing the absorbance-matched emission spectra in DMSO, demonstrating an increase in fluorescence with a restriction in rotational freedom upon binding of an analyte to a microbead construct of an embodiment of the present invention; and

FIGS. 4B and 4C are graphs showing the modes of detection for changes in optical signature upon binding of an analyte to a microbead construct of the present invention.

### DETAILED DESCRIPTION

In an embodiment, the present invention provides a microparticle assembly that overcomes, or at least ameliorates, one or more of the disadvantages described above.

In another embodiment, the present invention provides a microarray that overcomes, or at least ameliorates, one or more of the disadvantages described above.

According to a first embodiment, there is provided a bead construct, comprising:
(a) a bead;
(b) a fluorophore molecule, wherein the fluorophore molecule comprises at least two subunits connected to each other by at least one internal linker that provides conformational freedom to the at least two subunits, and wherein the optical signal emitted by the fluorophore changes when reducing the conformational freedom of the at least two subunits to each other;
(c) a first linker molecule connected to the bead and to one of the subunits of the flurophore molecule;
(d) a second linker molecule connected to another subunit of the fluorophore molecule; and
(e) a ligand molecule connected to the second linker.

According to a second embodiment, there is provided a microarray, comprising a plurality of bead constructs disposed thereon, wherein the bead constructs comprise:
(a) a bead;
(b) a fluorophore molecule, wherein the fluorophore molecule comprises at least two subunits connected to each other by at least one internal linker that provides conformational freedom to the at least two subunits, and wherein the optical signal emitted by the flurophore changes when reducing the conformational freedom of the at least two subunits to each other;
(c) a first linker molecule connected to the bead and to one of the subunits of the flurophore molecule;
(d) a second linker molecule connected to another subunit of the fluorophore molecule; and
(e) a ligand molecule connected to the second linker.

According to a third embodiment, there is provided a method for manufacturing a microarray, comprising:

depositing a plurality of bead constructs onto a

substrate, wherein the bead constructs comprise:
(a) a bead;
(b) a fluorophore molecule, wherein the fluorophore molecule comprises at least two subunits connected to each other by at least one internal linker that provides conformational freedom to the at least two subunits, and wherein the optical signal emitted by the flurophore changes when reducing the conformational freedom of the at least two subunits to each other;
(c) a first linker molecule connected to the bead and to one of the subunits of the flurophore molecule;
(d) a second linker molecule connected to another subunit of the fluorophore molecule; and
(e) a ligand molecule connected to the second linker.

According to a fourth embodiment, there is provided a method for identifying the presence of at least one target analyte in a sample, the method comprising:
providing a microarray as defined above;
incubating said microarray with said sample to thereby cause said at least one target analyte, if present in said sample, to bind to at least one ligand molecule, wherein the binding of said at least one target analyte reduces the conformational freedom of the at least two subunits of a flurophore molecule to each other, to produce a change in an optical signal; and
measuring said change in the optical signal, thereby identifying the presence of said at least one target analyte in said sample.

According to a fifth embodiment, there is provided a kit A kit for identifying the presence of a target analyte in a sample, the kit comprising:
a microarray according as defined above, one or more reagents, and instructions for use.

According to a sixth embodiment, there is provided a microparticle assembly for identifying the presence of a target analyte in a sample, the microparticle assembly comprising a microparticle coupled to a ligand and a plurality of moieties that are selected such that, if said target analyte is present in said sample, said target analyte interacts with said ligand and causes a change in the conformational freedom between said moieties that generates a change in an optical signal to identify said target analyte.

Advantageously, a reagent is not required in order to identify the presence of the target analyte in the sample. Hence, an additional reagent incubating step is not required. This may aid in minimizing the time and steps involved when identifying the presence of a target analyte in the sample.

Advantageously, a reagent-less and expeditious method for identifying a target analyte in a sample is provided.

Advantageously, a single incubation step that does not require any added reagent (that is, reagentless) is all that is needed to identify the presence of a target analyte in the sample. The disclosed microparticle assembly may further be used to form a microarray for multiplex analysis.

Advantageously, real time measurement of kinetic binding or reaction events between a target and the ligand can be obtained. In kinetic measurements performed using the microparticle assembly, a change in optical signature over time is recorded. Advantageously, this makes the measurement independent from any initial signal or background or unspecific signal.

According to a seventh embodiment, there is provided a microarray for identifying the presence of at least one target analyte in a sample, the microarray comprising a plurality of microparticle assemblies disposed therein, each microparticle assembly comprising a microparticle coupled to a ligand and a plurality of moieties that are selected such that, if said target analyte is present in said sample, said target analyte interacts with said ligand and causes a change in the conformational freedom between said moieties that generates a change in an optical signal to identify said target analyte.

According to an eighth embodiment, there is provided a method for manufacturing a microarray to identify the presence of a target analyte in a sample, comprising the step of:

depositing a plurality of microparticle assemblies onto the microarray, each microparticle assembly comprising a microparticle coupled to a ligand and a plurality of moieties that are selected such that, if said target analyte is present in said sample, said target analyte interacts with said ligand and causes a change in the conformational freedom between said moieties that generates a change in an optical signal to identify said target analyte.

According to a ninth embodiment, there is provided a method for identifying the presence of at least one target analyte in a sample, the method comprising the steps of:

providing a microarray having a plurality of microparticle assemblies disposed therein, each microparticle assembly comprising a microparticle coupled to a ligand and a plurality of moieties;

incubating the microarray with the sample to thereby cause at least one target analyte, if present in the sample, to interact with the ligand, wherein interaction of at least one target analyte changes the conformational freedom between the moieties to generate a change in an optical signal; and

measuring said change in the optical signal of the moieties to thereby identify the presence of at least one target analyte in the sample.

According to a tenth embodiment, there is provided a kit for identifying the presence of a target analyte in a sample, the kit comprising a microarray comprising a plurality of microparticle assemblies disposed therein, each microparticle assembly comprising a microparticle coupled to a ligand and a plurality of moieties that are selected such that, if said target analyte is present in said sample, said target analyte interacts with said ligand and causes a change in the conformational freedom between said moieties that generates a change in an optical signal to identify said target analyte; and instructions for use.

### DEFINITIONS

The following words and terms used herein shall have the meaning indicated:

The terms “microarray” or “array” as used herein refers to an array of microparticles that are capable of identifying the presence of a target analyte in a sample. A plurality of “microparticle assemblies” or “bead constructs” can form a microarray. The terms “microarray” or “array” includes “liquid arrays” or “planar arrays”.

The term “liquid arrays” refers to arrays in which microparticles or microbeads remain in suspension.

The term “planar arrays” refers to arrays in which microparticles or microbeads are deposited onto a planar substrate material.

The term “conformational freedom”, when used in conjunction with a pair of moieties, multiple moieties or moiety ligand assemblies refers to the spatial boundaries of these moieties due to the allowed movement of the moieties. The conformational freedom of the moieties can change due to spatial motion or rotational motion or spatial position of two or more moieties to each other or spatial position of moieties ligand assemblies to each other. Due to the change in the conformational freedom, the optical signature or property of the moieties can change accordingly. The moieties can be subunits of the same molecule or at least two separate molecules.

The term “interacts” refers to any contact, reaction, hybridization or binding that occurs between a target analyte and a ligand.

The term “bead” refers to a spherical particle which can be in the micron-sized range, e.g. from 1 micron to about 1000 microns, or from 1 micron to 10 microns, or from 10 microns to 100 microns, or from 100 microns to 1000 microns, and may also be referred to as a “microbead”; or in the nano-sized range (e.g. from 1 nm to 1000 nm, or from 1 nm to 10 nm, or from 10 nm to 100 nm, or from 100 nm to 1000 nm), and may also be referred to as a “nanobead”.

The term “microparticle” refers to a particle having a particle size in the micron-sized range, or from 0.1 microns to about 1000 microns. In one embodiment, when the microparticle is substantially spherical in shape, the particle size refers to the diameter of the microparticle, which is in the micron-sized range. Where the microparticle is spherical shaped, the “microparticle” is then termed as a “microbead”. In another embodiment, where the microparticle does not have a spherical shape, the particle size may refer to the equivalent diameter of the particle relative to a spherical particle or may refer to a dimension (length, breadth, height or thickness) of the non-spherical particle.

The term “microparticle assembly” or “microbead assembly” or “microbead construct” as used herein refers to microparticles or microbeads that are coupled to a plurality of moieties that are capable of generating an optical signature. Typically, the plurality of moieties may be connected to the microparticle by a first linker molecule. The moieties may be connected to each other by an internal linker molecule. The moieties may be connected to or bound with a ligand that is capable of binding the target analyte. The ligand may also be individually bound to the microbead. Due to the presence of the moieties present in the microparticle assembly or microbead assembly, the microparticle assembly or microbead assembly change their optical signal or optical property upon binding of a target analyte so as to advantageously enable the detection of the target analyte without the need of additional reagents.

The term “linker”, as used herein, refers to a chemical entity linking at least two other entities together. Typically, a linker uses covalent bonding. Typically, a linker could be a single bond or is composed from a number of 1 to 10 atoms forming the backbone of the linker.

The term “internal linker” refers to a linker between the moieties. The internal linker provides conformational freedom, for example, rotational freedom to the moieties relative to each other. The conformational freedom is changed, for example, increased or restricted upon binding of a target analyte to the ligand or other events involving the ligand such as ligand cleavage, hybridization events or other events in general causing a change in the molecular mass or stiffness of the moieties.

The term, “second linker” refers to a linker connecting a moiety to a ligand molecule. Typically the first and the second linker are linked to different moieties.

The term “organic polymer” as used herein, refers to a polymeric material which has repeating units of a backbone composed mainly of carbon atoms or a ring containing carbon atoms, and which also contains hydrogen. The polymeric material may also contain other elements such as, for example, sulphur, oxygen or nitrogen.

The term “moieties” refer to a part or subunit of a molecule or a distinct molecule. The molecule may be a fluorescent probe (also known as “fluorophore”), a dye (such as a quencher or reporter dye) or a quantum dot.

The term “population” as used herein refers to the total universe of microparticles.

The term “plurality of microparticles or microbeads or beads” as used herein refers to any number of microparticles; typically a larger number of microparticles.

The term “ligand” refers to any chemical agent that is chemically active or biological agent that is biologically active and which is capable of binding or reacting with a target analyte or an intermediary bound to the target analyte. The active agent may exhibit chemical activity and may include an environmental contaminant such as organic materials (for example, aliphatic hydrocarbon compounds, aromatic-containing compounds and chlorinated compounds) or inorganic materials (for example, metals and nitrates); a chemical warfare agent (for example, nerve agents such as sarin, soman, tabun and cyclosarin, blood agents such as arsines and hydrogen cyanide, or lachrymatory agents such as tear gas and pepper spray); a herbicide; a pesticide; a metabolite; a drug; lipids, carbohydrates, or a chemical catalyst. The active agent may exhibit biological activity and may be referred to herein as a “bioactive agent”. Exemplary bioactive agents include proteins, oligopeptides, small organic molecules, coordination complexes, aptamers, cells, cell fragments, virus particles, antigens, polysaccharides and polynucleotides, which can be attached to or bonded to a microparticle.

The term “target analyte” or “analyte” refers to a substance to be detected that is capable of binding to the ligand or is reacting with the ligand. A target analyte may also be a substance to be detected for calibration purposes. Exemplary target analytes include, but are not limited to, nucleic acids, polynucleotides, drugs, hormones, proteins, enzymes, antibodies, carbohydrates, receptors, bacteria, cells, virus particles, spores, and antigens.

The term “reagent” or “reagents” refer to a substance or a mixture of substances that is necessary to perform bioassays or microarrays. Typically, a reagent is a fluorescent labeled binding molecule, antibody, receptor or aptamer. Reagents can be mixed to create a mixture that can detect more than one target analyte.

The term “optical signature” or “optical property” as used herein refers to any optical signal or information which is obtained from the microparticle assembly by optical methods, such as fluorescence intensity, fluorescence spectra, optical spectroscopic, changes in absorption or emission maxima or minima, quenching, luminescence, chemo luminescence, Raman spectroscopy, UV-VIS spectroscopy or naked eye visualization. Of particular relevance to the present invention is an increase of fluorescence intensity, in particular this increase is caused by a reduced sterical freedom also referred to as “aggregation induced fluorescence”.

The term “protein” as used herein may be defined as two or more covalently bonded amino acid, which includes proteins, polypeptides, oligopeptides and peptides.

The term “reagentless” refers to a bioassay or microarray process, protocol or method that does not require the addition of any reagent or solution other than the analyte solution.

The terms “amino acid” and “peptide”, as used herein refer to both naturally occurring and synthetic amino acid and amino acid chains respectively.

The term “alkyl” used alone or as part of a larger moiety such as “haloalkyl”, or “alkylamine” refers to a straight or branched, saturated aliphatic group having the specified number of carbons, typically having 1 to 12 carbon atoms. More particularly, the aliphatic group may have 1 to 8, 1 to 6, or 1 to 4 carbon atoms. This term is exemplified by groups such as methyl, ethyl, n-propyl, isopropyl, n-butyl, isobutyl, tert-butyl, n-hexyl, and the like.

The term “alkylene” refers to a bivalent saturated straight-chained hydrocarbon, e.g., C₁-C₆ alkylene includes —(CH₂)₆—, —CH₂—CH—(CH₂)₃CH₃, and the like. The term “bivalent” means that the alkylene group is attached to the remainder of the molecule through two different carbon atoms.

The term “alkoxy” refers to the group —O—R where R is “alkyl”, “cycloalkyl”, “alkenyl”, or “alkynyl”. Examples of alkoxy groups include for example, methoxy, ethoxy, ethenoxy, and the like.

The term “amino” means —NH₂; while the terms “alkylamine” and “dialkylamine” mean —NHR and —NR₂, respectively, wherein R is an alkyl group.

The term “aryl” used alone or as part of a larger moiety means any aromatic or heteroaromatic group (both monocyclic and polycyclic moieties incorporating C, N, S, P, O, or not) and includes substituted phenyl, naphthyl, pyridyl, thiophenenyl, quinolinyl, triazolyl and the like.

The term “haloalkyl” includes mono, poly, and perhaloalkyl groups where the halogens are independently selected from fluorine, chlorine, bromine and iodine.

The term “cycloalkyl” used alone or as part of a larger moiety such as “cycloalkylalkyl” refers to a monocyclic or polycyclic, non-aromatic ring system of 3 to 20 carbon atoms, 3 to 12 carbon atoms, or 3 to 9 carbon atoms, which may be saturated or unsaturated. Examples of cycloalkyl groups include cyclopropyl, cyclobutyl, cyclopentyl, cyclohexyl, cyclohexenyl, cyclohexa-1,3-dienyl, cyclooctyl, cycloheptanyl, norbornyl, adamantyl, and the like.

The term “alkenyl” refers to a straight or branched aliphatic group with at least one double bond. Typically, alkenyl groups have from 2 to 12 carbon atoms, from 2 to 8, from 2 to 6, or from 2 to 4 carbon atoms. Examples of alkenyl groups include ethenyl (—CH═CH₂), n-2-propenyl (allyl, —CH₂CH═CH₂), pentenyl, hexenyl, and the like.

The term “alkynyl” refers to a straight or branched aliphatic group having at least 1 site of alkynyl unsaturation. Typically, alkynyl groups contain 2 to 12, 2 to 8, 2 to 6 or 2 to 4 carbon atoms. Examples of alkynyl groups include ethynyl (—C≡CH), propargyl (—CH₂C≡CH), pentynyl, hexynyl, and the like.

The term “functionalized” refers to the incorporation of chemical bonds such as amide, imide, carbamate, carbonate, ether, thioether, and the like, into the linker portion by using substituted functionality such as O, N, S, P, C═O to replace C, CH, CH₂ and CH₃ groups.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

### DETAILED DISCLOSURE OF EMBODIMENTS

Exemplary, non-limiting embodiments of a microparticle assembly for identifying the presence of a target analyte in a sample will now be disclosed.

The microparticle assembly comprises a microparticle coupled to a ligand and a plurality of moieties that are selected such that, if said target analyte is present in said sample, said target analyte interacts with said ligand and causes a change in the conformational freedom between said moieties that generates a change in an optical signal to identify said target analyte.

The change in the optical signal may be from no optical signal being present before interacting of the target analyte to the optical signal being present after interacting of the target analyte.

The change in the optical signal may be an increase in the level of optical signal after the interacting of the target analyte.

The change in the optical signal may be from a first optical signal being present before interacting of the target analyte to a second optical signal being present after interacting of the target analyte.

The change in the optical signal may be from an optical signal being present before interacting of the target analyte to no optical signal being present after interacting of the target analyte.

The change in the conformational freedom may be due to a change in the spatial distance between the moieties. The change in the conformational freedom may be due to a change in the orientation of the moieties. The reduction in conformational freedom may also be due to a change in the spatial distance between the moieties.

The microparticle may have a particle size in the range from about 50 nm to about 500 microns, from 0.1 microns to about 500 microns, from 50 nm to about 100 microns, or from about 1 micron to about 10 microns. If the microparticle is a spherical particle, the microparticle is then termed as a microbead. The microbead may be a capsule or hollow capsule. The microparticle may be a polymeric microparticle. The microparticle may be made from a material selected from the group consisting of organic polymers (such as but not limited to polystyrene, hydrogel, agarose, alginate), melamin, organic crystals or metallic microparticles (such as but not limited to gold, silver or iron oxide), inorganic microparticles (such as but not limited to silicon dioxide, titanium dioxide, glass or quantum dots) or mixtures thereof.

The microparticle may be connected to the moieties via a first linker molecule. In order to limit the rotational freedom of the moiety attached to the first linker molecule, the first linker molecule may terminate in a sterically demanding anchor group to which the moiety is attached. This approach will eliminate rotational freedom for the moiety to relax. The first linker molecule may be about 0.2 to about 500 nm long. The first linker molecule may be an organic polymer. The organic polymer may be selected from the group of polymers consisting of poly ethylene glycol (PEG), poly(vinylpyrrolidone) (PVPON), oligo (ethylene glycol), polyacrylamide, dextrane, poly-2-methyl-2-oxazoline (PMOXA), zwitterionic coatings (such as poly(carboxybetaine methacrylate)) and other synthetic polymers containing these polymers such as poly(L-lysine) (PLL)-graft-PEG, degradable or non-degradable polymers (such as poly(styrenesulfonate), polyanhydride, poly(acrylic acid), polyethyleneimine, polyglycolic acid and polycaprolactone) and dextran sulfate.

The moieties may include a number of subunits that make up one molecule or may be a plurality of molecules such as a pair of moieties. The molecule may have at least one moiety that provides conformational freedom to at least two different structural parts of the molecule and emitting light as a function of the conformational orientation of at least two structural parts of the molecule to each other. The optical property of the moieties may be affected by changes in the conformation and relative position of these moieties to each other. The changes in conformation are caused by changes to the conformational freedom of the molecule.

The moieties may be selected from the group consisting of a metal complex, a quantum dot, a fluorescent probe, a fluorescent protein, a reporter dye, a quencher dye, chromophore, rare earth complex, spin-probe, luminophore, N,N′-diphenyl-N,N′-bis(3-methylphenyl)-(1,1′-biphenyl)-4,4′-diamine (TPD), silole, tetraphenylethene (TPE), parts thereof and derivatives thereof.

The moieties may be subunits of one fluorophore molecule or may be a number of distinct fluorophore molecules such as two fluorophore molecules. When the moieties are a number of subunits, the subunits may be linked together by an internal linker formed by H-bonds, salt bridges, electrostatic interaction, at least one covalent bond or at least one ionic bond. Typical examples in which the moieties are subunits of one fluorescent molecule are siloles and tetraphenylethene (TPE) derivatives.

The moieties may be a quantum dot metal particle pair. The quantum dot may emit considerable more light when the metal particle is in close proximity to the quantum dot due to surface plasmon resonance phenomena. The pair of moieties or assembly of moieties may also consist of the same chemical entity. The pair of moieties may consist of one fluorescent protein and one fluorophore or may be two fluorescent proteins. The two fluorescent proteins may act as bulky entities forcing the conformation into a state with a large distance between the two fluorescent proteins or the fluorescent protein and another fluorophore type. At this conformational stage, the distance is large and no interaction is observed. The pair of moieties may be a quencher-dye pair or a FRET dye pair.

The fluorophore molecule may be selected from one or more of the following classes in racemic, chiral and/or non-chiral forms: chemo- or bioluminescent molecules (such as Rhodamine, Fluorescein, Coumarin, Acridin, Bodipy, Dansyl, Dapoxy, Hoechst, Cy, merocyanines, DAPI, SYTO, Alexa Fluor dyes, Pechmann dyes, polymethines, biaryl or bi(heteroaryl) dyes, thiophenes, helicines, phenylenes, xanthenes, naphthylenes and their derivatives, isomers and/or conjugates thereof), photochromic molecules (such as spiropyrans, spirooxazines, quinones, diaryethenes, azobenzenes, and their derivatives, isomers and/or conjugates thereof), aggregation-induced emitters (e.g. tetraphenylethene, hexaphenylsilole, diphenyldibenzofulvene, siloles, and their derivatives, isomers and/or conjugates thereof), aggregation-quenched fluorophores, aggregation induced fluorescent fluorophores, black-hole quenchers paired with regular fluorophores, fluorescent proteins (GFP, RFP, YFP, CFP and constructs thereof), FRET dye pairs (e.g. mOrange2/mCherry, CFP/YFP, FITC/TRITC, etc.), metal-enhanced fluorophores (such as metal-ligand complexes based on porphyrins, bipyridyl, phenanthrolines, e.g. Ru(bpy)₂(dcbpy) (PF₆)₂), stacking-induced emission or quenching (e.g., derivatives, isomers and/or conjugates thereof of perylene, pyrene, terylene, etc.), Dexter and Exciplex quenchers, etc. The fluorophore molecule may be an aggregation-induced fluorophore, a mono- or a di-component fluorophore.

An internal linker molecule may be used to connect a structural part of the molecule different to the structural part of the molecule to which the first linker molecule is connected to. The internal linker molecule may provide a large degree of conformational freedom resulting in a typical fluorescence intensity. The internal linker molecule allows the moieties to have rotational freedom such that the moieties can have any rotational freedom between 0 and 360 degree.

The second linker typically connects the ligand to a moiety different to which the first linker is connected. The second linker molecule may be a short and/or stiff (conformationally locked or restricted) linker allowing direct transfer of the additional steric hindrance, as imposed on the microparticle assembly upon binding of analyte molecules, to the ligand. A linker of this design will directly translate any binding event into a restriction of the rotational and conformational freedom of the moieties, thus resulting in a change of the fluorescence signal and optical signature. The second linker may be formed from a single covalent bond.

The first and/or second linker molecule may be one of the following. The first and/or second linker molecule may be a covalent bond, a functionalized PEG (CH₂CH₂O)ₙ, an alkyl group (CH₂)ₙ, an alkylene group, an aryl group (e.g., substituted phenyl), an alkenyl group, an alkoxy group, a haloalkyl group, an aminoalkyl group, a cycloalkyl group, an alkenyl group, an alkynyl group, a metal complex, etc.

The first and/or second linker molecule may be an alkylene group i.e., —(CH₂)ₓ—, where x is a positive integer (for example, from 1 to about 18, more specifically between 1 and about 10, or more specifically between 2 and 8, or more specifically between 4 and 8.) Any substitutable hydrogen on a carbon atom in the linker may be substituted by halo, OH, NO₂, CN, COOH, COO(C₁-C₆alkyl), amino, (C₁-C₆)alkyl, (C₁-C₆)alkoxy, (C₁-C₆)alkylamino, or di(C₁-C₆)alkylamino, wherein the (C₁-C₆)alkyl and (C₁-C₆)alkoxy in any of the preceding groups may be optionally substituted with halogen, (C₁-C₆)alkoxy, COOH, COO(C₁-C₆alkyl), OH, NO₂ or CN. In addition, the carbon chain of the alkylene linker can be optionally substituted with one or more groups including an alkene, alkyne, phenylene, ether, thioether, amine, (C₁-C₆)alkylamino, di(C₁-C₆)alkylamino, ester, thioester, amide or carbamate. In one embodiment, the first and/or second linker molecule may be an alkylene group that is substituted with one or more amide groups. The alkylene group may be substituted with two amide groups.

The first and/or second linker molecule may be represented by the following structural formula (I)

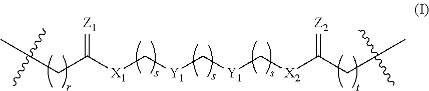

wherein each r, s, and t are independently and optionally 0, 1, 2, or 3, w is 1, and X₁, X₂, Y₁ and Y₂ are independently CR₂, O, S, or NR, wherein R is an alkyl group, an acyl group, an aryl group, a cycloalkyl group, or a heterocycle group. Alternatively, R can form an aryl group, a cycloalkyl group or a heterocycle group with another carbon atom within the linker. Z₁ and Z₂ is independently O, S or H₂.

The first and/or second linker molecule may be represented by the following structural formula (II)

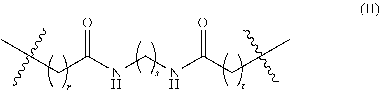

wherein r, s, and t are independently and optionally 0, 1, 2, or 3.

The first and/or second linker molecule may be represented by the following structural formula (III)

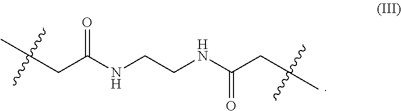 [SMILES: *CC(=O)NCCNC(=O)C*]

The first and/or second linker molecule may be represented by the following structural formula (IV)

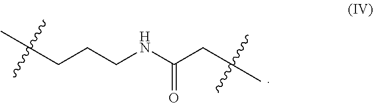 [SMILES: *CCCNC(=O)C*]

The first and/or second linker molecule may be represented by the following structural formula (V)

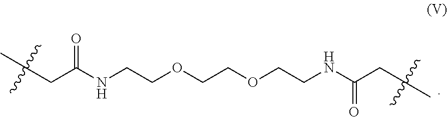 [SMILES: *CC(=O)NCCOCCOCCNC(=O)C*]

The squiggly line ( [symbol] ) in the above structural formulae (I) to (V) indicates the point of connection to the moiety and the microparticle or other moieties. Either the moiety or the microparticle can be attached to either the left side or the right side of the linker molecule. Any substitutable hydrogen on a carbon atom of this linker molecule may be substituted by halo, OH, NO₂, CN, COOH, COO(C₁-C₆alkyl), amino, (C₁-C₆)alkyl, (C₁-C₆)alkoxy, (C₁-C₆)alkylamino, or di(C₁-C₆)alkylamino, wherein the (C₁-C₆)alkyl and (C₁-C₆)alkoxy in any of the preceding groups may be optionally substituted with halogen, (C₁-C₆)alkoxy, COOH, COO(C₁-C₆alkyl), OH, NO₂ or CN. Any substitutable hydrogen on a nitrogen atom may be substituted by 1 to 3 groups selected from (C₁-C₆)alkyl, optionally substituted with halogen, (C₁-C₆)alkoxy, COOH, COO(C₁-C₆alkyl), OH, NO₂ or CN. In a specific embodiment, r and t are independently 1 or 2 and s is 2 or 3.

To render the microparticle surface with functional groups to attach the first linker molecule, and give the microparticle antifouling properties, any form of functional coating can be applied by one of the following techniques: wet chemical deposition, chemical vapor deposition, physical vapor deposition, chemical and electrochemical techniques, spraying and variations thereof. A functional coating herein comprises coatings that can be further functionalized with the linker and/or moieties, i.e. that have some form of reactive surface functionality.

The ligand may interact with the target analyte by binding to the target analyte, by cleavage or removal of the ligand by the target analyte as well as hybridization of the target analyte. The ligand may be connected to the second linker molecule. The ligand may be connected to one of the moieties via a third linker molecule. The ligand may be connected directly to the microparticle. Typically, the ligand does not limit the conformational freedom, such as free rotation or other conformational changes in the moieties. Upon binding of a target analyte molecule (which is typically larger than the ligand) to the ligand, the conformational freedom (including spatial freedom or rotational freedom) is reduced, causing a direct change in fluorescence intensity and spectroscopic profile that are proportional to the binding event. By comparing and/or monitoring the optical properties before and after the addition of the sample containing target analytes, the presence of a target analyte is detected. By further determining the intensity in change of the optical property, a quantitative measurement of the analyte concentration is performed. Typically the absolute or relative changes in optical signature are first measured using standard reference materials of the analytes to obtain a calibration.

The ligand may be selected from the group consisting of a drug, a metabolite, a peptide, an antigen, a biotin, a hormone, a steroid, a toxin, an antibiotic, an allergen, a protein, a cofactor, pesticide, insecticide, oligonucleotide, polymer, biopolymer, saccharide, lipid, a metal, an electrochemically reactive ligand such as a cytochrome, parts thereof, derivatives thereof and mixtures thereof.

Methods to conjugate linker molecules with the moieties, ligands and/or microparticles can be based on all standard chemical conjugation (ligation) methods such as amide, ester, carbamate, carbonate, disulphide, maleimide, thioester, Staudinger-based, nucleophilic substitution-based (e.g., aromatic or aliphatic displacement methods), diazo-based, radical-based, carbene-based, cross metathesis-based (e.g., alkyne and alkene based) and cycloaddition-based (e.g., Huisgen 1,3-dipolar azide-alkyne ‘click’-based) chemistry.

The target analyte may be an organic or an inorganic molecule. The target analyte may be selected from the group consisting of environmental pollutant (including pesticide, insecticide, toxin, etc.); a chemical (including solvent, polymer, organic material, metal ion, etc.); therapeutic molecule (including therapeutic and abused drug, antibiotic, etc.); biomolecule (including hormone, cytokine, protein, nucleic acid, lipid, carbohydrate, enzyme, antibody, antigen, cellular membrane antigen and receptor (neural, hormonal, nutrient, and cell surface receptor); whole cell (including prokaryotic (such as pathogenic bacteria) and eukaryotic cell, including mammalian tumor cell); virus (including retrovirus, herpesvirus, adenovirus, lentivirus, etc.); and spore. The target analyte may be a nucleic acid or a protein (including immunoglobulin; enzyme, hormone and cytokine). The specific binding of the target analyte to the ligand may be dependent on chemical interactions selected from the group consisting of electrostatic interaction, ionic bonds, covalent bonds, hydrogen bonds and dipole-dipole interactions.

In another preferred embodiment, the microparticle assembly comprises a microparticle coupled to a ligand and a plurality of moieties that are selected such that, if said target analyte is present in said sample, said target analyte interacts with said ligand and causes a change in the conformational freedom between said moieties that generates a change in an optical signal to identify said target analyte. The moieties and ligand may be individually bound to the microparticle via a first linker. The surface density of moieties and ligand may be selected such that interaction of the target analyte will reduce the available space for moieties and thereby reducing the conformational freedom of moieties by reducing the distance of moieties to each other causing a change in optical signature.

The following design rules are in general applicable when forming a microparticle assembly but should not be interpreted as limiting the invention. The distance between a donor and acceptor fluorophore molecule at which the energy transfer is 50% efficient is defined by the Foerster radius (R₀), wherein the magnitude of R₀ depends on the spectral properties of the donor and acceptor dyes. The first fluorophore could be the donor fluorophore molecule and the second fluorophore could be the acceptor fluorophore molecule, or vice versa. The FRET efficiency (E) is the quantum yield of the energy transfer transition occurring per donor excitation event. The FRET efficiency depends on several parameters such as the distance between the donor and the acceptor; the spectral overlap of the donor emission spectrum and the acceptor absorption spectrum and the relative orientation of the donor and acceptor molecule. The FRET efficiency depends very strongly on the donor-to-acceptor separation distance (r), and increases with reduced distance by the 6th power law, due to the dipole-dipole coupling mechanism of FRET. At a distance of r greater than 2×R₀, almost no FRET occurs; strong FRET occurs at distances of 0.5 R₀. The design rules for the second linker molecule, linking the two fluorophore molecules is thus: a large distance (r) should be achieved when no binding of the target analyte onto the ligand takes place. This non-binding distance (r) is preferentially larger than 2×R₀. The distance (r) should be reduced after the binding event. This binding-event distance (r) is preferentially smaller than R₀. Because R₀ can be tuned by the selection of the donor-acceptor pair and (r) can be tuned by the linker design, optimization of R₀ and (r) will produce the most efficient bead construct.

To effect changes in the photo-physicochemical properties (i.e., optical signature changes) of the moieties, several fundamental design elements are conceivable. These comprise, but are not limited to: restriction of conformational freedom through steric, dipole-dipole, reduction of available volume or space, ionic, metal-ligand-field, oxidation, conjugation states, electrostatic, pi-stacking, aggregation and hydrogen-bonding events; pH-dependent extension or stoppage of pi-conjugation through changes in protonation or chemical states in either cyclized or linear forms; external changes in ligand-binding field effects for metal complexes through external alterations in energy quenching, energy transfer (conversion) and/or energy enhancement events.

When the microparticle assembly, or microarray formed from the microparticle assembly(ies), are being used to analyze a sample that potentially contains a target analyte, a change in optical property is recorded as a result of the interaction between the ligand and the target analyte. In general, the change in optical property can be observed continuously and therefore referred as a kinetic measurement or, after a predetermined time, referred as an end-point measurement. The optical property of the microparticle assembly may be detected with a detector, such as an optical detector. The optical detector may send a signal to the computer memory, which is then accessed by a computer processor for generating an image file. Data associated with the image file such as the position; type of microparticle and optical properties, optical spectra's and optical intensity is then used to determine an analyte or multiple analytes in a sample. The presented method enables qualitative and quantitative analysis.

Accordingly, there may be provided a microarray system for identifying the presence of one or more target analytes in a sample comprising: a microarray comprising a plurality of microparticle assemblies, each carrying a different ligand for multiplex bioassays; a detector to detect optical properties originating from the microparticle assemblies upon interaction between the target analyte molecules and the respective ligands of the microparticle assembles; and a processor responsive to program instructions to receive data from the detector to identify the presence of the one or more target analytes in the sample.

The disclosed process may enable microparticle arrays with high multiplexing capabilities to be generated.

In the following, different types of bioassays that can be performed using the microparticle assembly are disclosed. In general, any biological interaction of the ligand that causes a change in molecular weight or stiffness can be detected by the microparticle assembly. The following types of bioassays are described in the following: a) binding assays, b) enzymatic assays based on cleavage, c) enzymatic assays based on polymerization.

In one embodiment, the microparticle assembly is used to perform binding assays. In this assay format, a target analyte molecule binds to the ligand of the microparticle assembly and increase the molecular weight and/or stiffness. Both events will cause a reduction in conformational freedom resulting in a change in optical signature.

In another embodiment, the microparticle assembly is used to perform enzymatic assays. In this assay format, the ligand may act as an enzyme substrate or co-substrate. Here, the ligand may be a peptide and the analyte enzyme may be a peptidase. Upon interaction of the enzyme with the ligand, the ligand is cleaved and is partially or completely removed causing a reduction in molecular weight and/or stiffness. By variation of the peptide sequence, bioassays specific for particular proteases can be designed. Both events will cause an increase in the conformational freedom resulting in a change in optical signature. In another example, the ligand may be a single strand DNA molecule and may act as a template. Upon contact with a primer, DNTPs and a polymerase, a complementary DNA strand is formed on the ligand causing an increase in molecular weight and stiffness. Both events will cause a reduction in conformational freedom resulting in a change in optical signature. In general, enzymatic assays for hydrolases, synthetases, oxidoreductases, topomerases, transferases, lyases, isomerases and ligases can be designed as long as the molecular weight, conformational change or change in stiffness is caused by the enzyme on the ligand.

The microarray may be used for screening a number of samples. First, several batches of microparticle assemblies carrying different low molecular drug molecules as ligands are prepared. Second, a planar bead microarray is formed; encoding of the bead constructs can be done by existing methods such as spatial encoding technology. Third, the planar bead array is observed by a detector to detect changes in optical property; typically this is a change in fluorescent intensity. By using this screening technology, array biomolecules with a binding affinity to any of the present and known ligands can be detected. No reagent or additional labeling step is necessary; keeping the sample molecules in its native state. A change in optical property is directly observed upon sample addition and binding to ligands. Optional sample molecules can than be sequentially washed off and collected for further studies by the addition of the respective ligand. In this way, a high throughput screening of potential drug targets combined with the isolation is performed.

There is also provided a microarray for identifying the presence of at least one target analyte in a sample, the microarray comprising a plurality of microparticle assemblies disposed therein, each microparticle assembly comprising a microparticle coupled to a ligand and a plurality of moieties that are selected such that, if said target analyte is present in said sample, said target analyte interacts with said ligand and causes a change in the conformational freedom between said moieties that generates a change in an optical signal to identify said target analyte.

Each microparticle assembly may carry a ligand that is different from each other so as to identify the presence of more than one target analytes in the sample.

There is also provided a method for manufacturing a microarray to identify the presence of a target analyte in a sample, comprising the step of:

depositing a plurality of microparticle assemblies onto the microarray, each microparticle assembly comprising a microparticle coupled to a ligand and a plurality of moieties that are selected such that, if said target analyte is present in said sample, said target analyte interacts with said ligand and causes a change in the conformational freedom between said moieties that generates a change in an optical signal to identify said target analyte.

There is further provided a method for manufacturing a microarray, comprising: depositing a plurality of bead constructs onto a substrate, wherein the bead constructs comprise:
(a) a bead;
(b) a fluorophore molecule, wherein the fluorophore molecule comprises at least two subunits connected to each other by at least one internal linker that provides conformational freedom to the at least two subunits, and wherein the optical signal emitted by the flurophore changes when reducing the conformational freedom of the at least two subunits to each other;
(c) a first linker molecule connected to the bead and to one of the subunits of the flurophore molecule;
(d) a second linker molecule connected to another subunit of the fluorophore molecule; and
(e) a ligand molecule connected to the second linker.

The substrate is any material which is capable of supporting the microparticles or beads of the present invention thereon and may include: polymeric materials, organic materials, inorganic materials, metals, ceramics, plastic, rubber, glass, fibrous materials, graphite or silicon, silicon dioxide, silicon nitride, modified silicon, glass, modified or functionalized glass, inorganic glass, plastics, acrylics, polystyrene, copolymers of styrene, polypropylene, polyethylene, polybutylene, polyurethane, Teflon, polysaccharide, nylon, nitrocellulose, resins, silica, silica-based materials and carbon.

There is also provided a method for identifying the presence of at least one target analyte in a sample, the method comprising the steps of:

providing a microarray having a plurality of microparticle assemblies disposed therein, each microparticle assembly comprising a microparticle coupled to a ligand and a plurality of moieties;

incubating the microarray with the sample to thereby cause at least one target analyte, if present in the sample, to interact with the ligand, wherein interaction of at least one target analyte changes the conformational freedom between the moieties to generate a change in an optical signal; and

measuring the change in the optical signal of the moieties to thereby identify the presence of at least one target analyte in the sample.

There is also provided a kit for identifying the presence of a target analyte in a sample, the kit comprising a microarray comprising a plurality of microparticle assemblies disposed therein, each microparticle assembly comprising a microparticle coupled to a ligand and a plurality of moieties that are selected such that, if said target analyte is present in said sample, said target analyte interacts with said ligand and causes a change in the conformational freedom between said moieties that generates a change in an optical signal to identify said target analyte; and instructions for use.

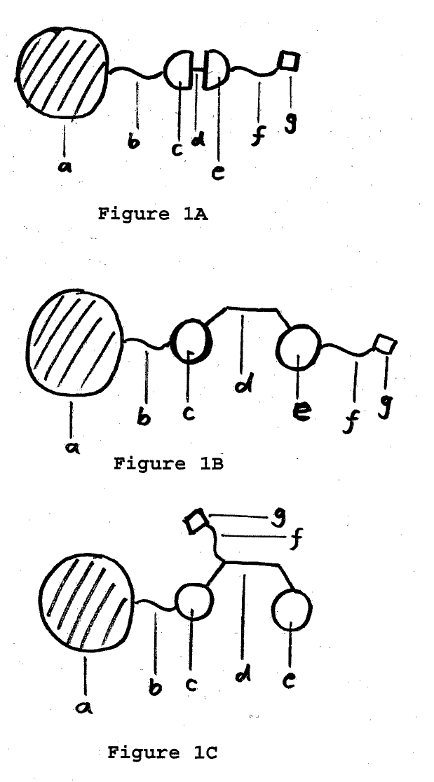

FIG. 1A depicts a microbead construct of the present invention, wherein changes in the conformation and relative position of at least two parts or subunits of a single fluorophore molecule to each other result in a change in the optical signature obtained (only two subunits shown in FIG. 1A for simplicity; a=microbead, b=first linker, c=first subunit (or moiety) of fluorophore, d=internal linker, e=second subunit (or moiety) of fluorophore, f=second linker, g=ligand).

In FIG. 1A, upon binding of a typical high molecular-weight analyte molecule to the ligand, the conformational freedom is restricted. This results in a change in conformation and a different characteristic optical signature is obtained compared to when no analyte molecule is bound to the ligand.

The microbead constructs shown in FIGS. 1B and 1C illustrate another two possible embodiments of the present invention, wherein each microbead construct has two fluorophores instead of a single fluorophore (a=microbead, b=first linker, c=first fluorophore, d=internal linker, e=second fluorophore, f=second linker, g=ligand). However, the two fluorophore microbead constructs function in a similar fashion to the single fluorophore microbead construct, i.e. upon binding of an analyte molecule, a change in steric or conformational freedom is caused, which results in a change in the optical signature obtained.

The primary difference between the microbead constructs of FIGS. 1B and 1C is that the second linker of the microbead construct is attached to the second fluorophore in FIG. 1B, whereas the second linker is attached to the internal linker in FIG. 1C. Nonetheless, in both microbead constructs, the rotational freedom is via the internal linker and the two fluorophore molecules can have any rotational freedom between 0 to 360 degrees. Upon binding of a high molecular weight analyte, the rotational freedom of the microbead constructs is substantially restricted, which results in a change in the optical signature obtained.

One of the principles that should be applied to the design of a two fluorophore microbead construct (as exemplified in FIGS. 1B and 1C) is that the donor-to-acceptor distance between the two fluorophores should be large when the target analyte is not bound to the ligand of the microbead construct, and should be subsequently reduced after the target analyte is bound. The donor-acceptor distance should thus preferentially be twice that of the Förster radius (R₀) before the binding event, and smaller than R₀ after the target analyte is bound to the ligand of the microbead construct.

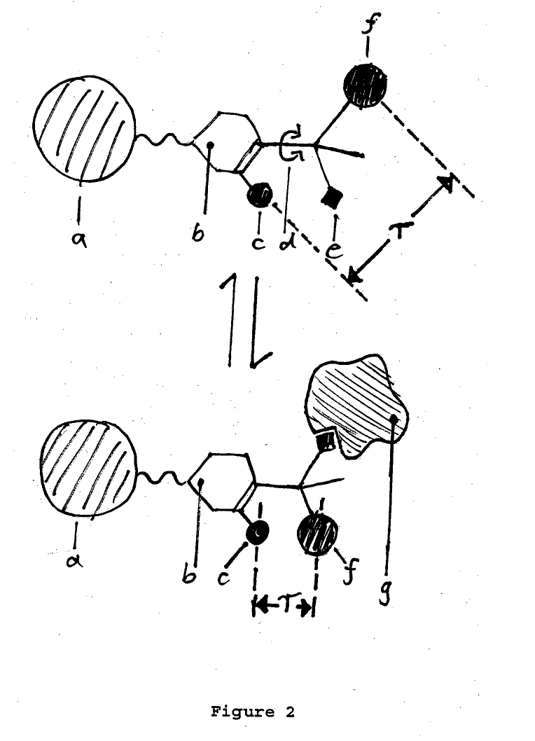

FIG. 2 is a schematic diagram which illustrates the above design principle [a=microbead, b=rigid part of the first linker, c=first fluorophore (or quencher, or metal particle or first subunit of a single fluorophore), d=rotational free part of the inner linker, e=ligand, f=second fluorophore (or quencher, or metal linker or second subunit of a single fluorophore), r=distance between the two fluorophores or fluorophore quencher pair or subunit pair, arrow=axis of rotational freedom].

In FIG. 2, the two fluorophores (or subunits) “c” and “f” are bulky and therefore at the state with lowest energy, the distance between the fluorophores (or subunits) “r” is large, resulting in no fluorescence (above). The analyte binding onto the ligand creates an even bulkier group and is forced away from the first fluorophore “c”. At the same time, this brings the second fluorophore “f” much closer to the first fluorophore “c” (much smaller “r” bellow R₀) causing fluorescence or quenching (depicted below). In this example, the ligand can also be directly linked to the second fluorophore or subunit. It is preferable if the signal difference (for example, the fluorescence intensity) is large between the non-binding and the binding-event.

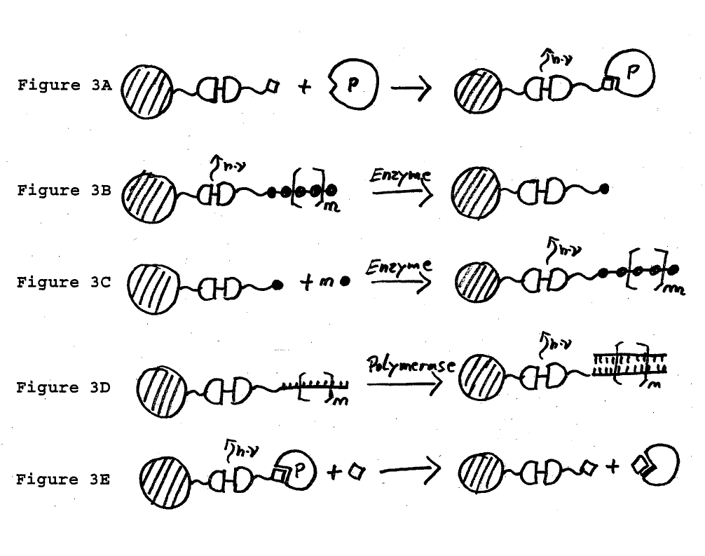

FIG. 3A is a schematic diagram which shows a bead bioassay involving the use of a microbead construct of the present invention, wherein the binding of a protein or other high molecular weight molecule to the ligand of the construct results in an increase in molecular weight and/or stiffness (P=protein or other high molecular weight molecule). This in turn causes a reduction in conformation freedom and a change in the optical signature obtained.

FIG. 3B is a schematic diagram which illustrates a bead bioassay involving the use of a microbead construct of the present invention, wherein the ligand is a protein or DNA or other polymer and a reduction in molecular weight and/or stiffness is caused by the cleavage of the protein by an analyte protease or peptidase, or cleavage of the DNA by an analyte DNAse (m=number of monomers). This in turn causes an increase in conformation freedom and a change in the optical signature obtained.

Alternatively, the ligand can also be a polyester or a polyamide or a polysaccharide, wherein a reduction in molecular weight and/or stiffness is caused by the hydrolysis of the polymer. This in turn causes an increase in conformation freedom and a change in the optical signature obtained.

FIG. 3C is a schematic diagram which illustrates a bead bioassay using a microbead construct of the present invention, wherein the ligand is a monomeric subunit or a small polymer and an increase of molecular weight is caused by the extension of the ligand.

FIG. 3D is a schematic diagram which illustrates a bead bioassay using a microbead construct of the present invention, wherein the ligand is a single stranded nucleic acid such as DNA or RNA and an increase in molecular weight and/or stiffness is caused by the formation of a complementary nucleic acid strand onto the ligand strand (m=number of monomers). This in turn causes a reduction in conformation freedom and a change in the optical signature obtained.

FIG. 3E is a schematic diagram which illustrates a bead bioassay using a microbead construct of the present invention using a mechanism opposite to FIG. 3A, wherein a large molecule bound to the ligand is displaced by the analyte reducing the molecular weight.

In all of the above examples of the bead bioassays, the optical signature may be continuously monitored.

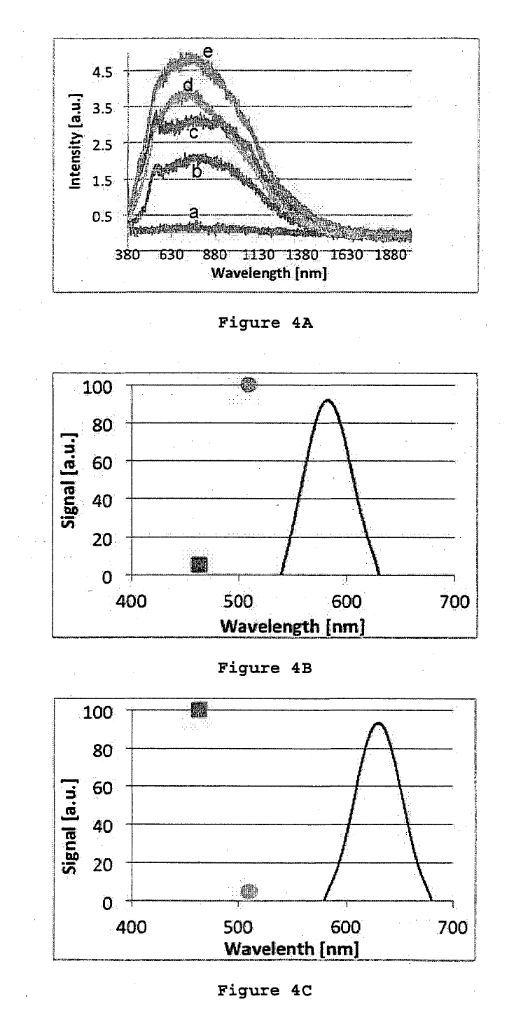

FIG. 4A is a graph which shows the absorbance-matched emission spectra in DMSO, thereby demonstrating an increase in fluorescence with a restriction in rotational freedom upon binding of an analyte to a microbead construct of the present invention. a=pure DMSO, b=tetraphenylethene bisazide (TPE bisazide), c=doubleclicked TPE with alkyne-functionalized Biotin, d=monoclicked TPE with alkyne-functionalized Biotin (in which a biotin molecule is attached (clicked) onto the fluorescent molecule TPE), e=double-clicked biotin modified TPE after streptavidin binding (in which two biotin molecules are clicked onto the fluorescent molecule TPE). The attachment (or clicking) of the biotin molecule(s) onto the fluorescent molecule TPE occurs due to a method called “click chemistry”, in which a chemical bond is formed between two chemical entities, one with an alkyne group and the other with an azide group. The chemical reaction is called an “Azide-Alkyne cycloaddition”.

FIGS. 4B and 4C are graphs which show the modes of detection for changes in optical signature upon binding of an analyte to a microbead construct of the present invention. Square series=increase in signal upon binding tetraphenylethenes (TPE)/Black Hole Quencher (BHQ) dye pairs, Circle series=signal reduction using the FRET phenomenon. Line=spectral shift of Seminaphtharhodafluor (SNARF) dye.

### EXAMPLES

Non-limiting examples of the invention and a comparative example will be further described in greater detail by reference to specific Examples, which should not be construed as in any way limiting the scope of the invention.

### Example 1

This example demonstrates the synthesis of a bead construct.

Microbead preparation: Carboxylated silica beads (5.64 μm±0.27 μm) were purchased from Microparticles GmbH (Berlin, Germany) and used as received. These beads may be substituted with particulates of any size (within the range indicated above) and material (such as listed earlier), either commercially available or synthetic (home-made).
Linker chemistry: For this example, propargylamine was attached to the surface of EDC/NHS-activated carboxylated silica beads. 200 microliter of bead stock solution was incubated with 1 mg/mL of EDC and 1 mg/mL NHS in PBS buffer (pH 6, Vivantis) for 2 h. After washing the beads by sequential centrifugation/redispersion cycles (3×), beads were dispersed in a solution of Propargylamine (2 mg/mL) in PBS (pH 8) for 2 h. Finally, non-reacted Alkyne was washed out by centrifugation and redispersion of the beads in DMSO (for further reactions) or water (for storage).
Fluorophore: As an example, a bi-functional click linker tetraphenylethene (TPE) was synthesized according to a literature protocol (Adv. Funct. Mater. 2009, 19, 1891-1900)). The bisazide click linker was then attached to the beads by exposing them to a 2 mg/mL TPE in DMSO in the presence of 2 mg/mL Copper Sulfate and 5 mg/mL Sodium Ascorbate in minute quantities of water using click chemistry.
Ligand: The specific interaction between biotin and streptavidin was used to demonstrate analyte binding as a proof of concept. Alkyne-functionalized Biotin acid was attached to the fluorophore via click chemistry. This facile step of pre-functionalizing the ligand with a click group allows for the attachment of second linker (short alkyl chain) and ligand (biotin) in one step.

### Example 2

This example demonstrates a binding assay using the bead construct and a conventional fluorescent spectrometer or fluorescent reader. Beads of the current invention were prepared comprising a ligand that can undergo a binding event for the detection of an analytes and were dispersed in 0.01 M PBS buffer. An aliquot of the bead suspension was pipetted into a cuvette and the fluorescence intensity was measured (baseline). An aliquot of the target analyte sample solution was added and the change in fluorescence intensity was continuously measured or recorded after 1 minute (measurement). A control measurement of the sample solution for intrinsic fluorescence was performed. A calibration curve was generated by repeating the above protocol with standard reference materials of the analyte. The analyte concentration of the sample was determined by comparing the change in fluorescence intensity of the sample with the calibration curve.

### Example 3

This example demonstrates multiplex detection by forming a bead microarray from sub-batches of the bead construct.

Bead assembly on planar substrate: Several batches of beads of the current invention were prepared, comprising different ligands for the detection of several different analytes and are dispersed in 0.01 M PBS buffer. A bead microarray was formed by assembling the beads in a random fashion on a planar substrate as taught in PCT/SG2007/000232 “A Microarray System and a Process for Producing Microarrays”. By applying several batches sequentially a microarray carrying sub-populations of beads was formed. Beads were identified by their spatial position on the microarray.
Bioassay protocol: An aliquot of the target analyte sample solution was added and incubated for 3 to 30 minutes. After incubation the array was washed with a washing buffer, e.g. 0.1 M PBS.
Optical imaging: The array was placed under a fluorescent microscope and the fluorescence intensity is measured (end point method). Alternative changes in fluorescent intensity are measured continuously (kinetic method). A calibration curve was generated by repeating the above protocol with all standard reference materials of the analytes. The analyte concentration of the sample was determined by comparing the change in fluorescence intensity of the sample with the calibration curve.

### Example 4

The following is a non-limiting example for the synthesis of compounds exhibiting aggregation induced fluorescence with functional groups on two moieties of the compound to attach linkers is given. Compound 8 is an example carrying a first and a second linker for coupling to a microbead and a ligand respectively.

### Synthesis and Chemical Analysis of the Fluorophores Amine TPE:

4-Aminebenzophenone (1, 400 mg, 2.03 mmol) was dissolved in THF (15 ml). The solution was cooled to 0° C. and Zn dust (332 mg, 5.07 mmol, 2.5 equiv.) and TiCl₄ (1 M in toluene, 4.06 ml, 4.06 mmol, 2 equiv.) were added. After refluxing for 4 h, 50 ml of a 10% K₂O₃ solution was added and stirred overnight at room temperature. The white precipitate (2) was extracted 3× with DCM.

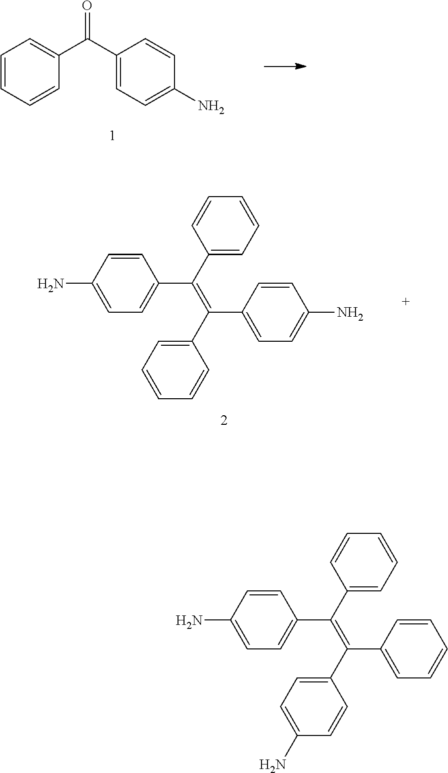 [SMILES: Nc1ccc(C(=O)c2ccccc2)cc1 ; Nc1ccc(/C(=C(\c2ccccc2)c2ccc(N)cc2)c2ccccc2)cc1 ; Nc1ccc(/C(=C(/c2ccccc2)c2ccc(N)cc2)c2ccccc2)cc1]

### Analysis of 2:

1H-NMR (400 MHz, CDCl₃): δ (ppm)=7.26-7.06 (m, 10H, Harom), 6.67-6.76 (m, 4H, Harom), 6.41-6.39 (m, 4H, Harom), 3.54 (s, 4H, Hamine)

### Carboxyl TPE

4-Methylbenzophenone (3, 2.0 g, 10.2 mmol, 1.0 equiv.) was dissolved in THF (25.5 ml) under ni-trogen. The solution was cooled to −78° C., and TiCl₄ (1 M in toluene, 12 ml, 12.0 mmol, 1.2 equiv.) and Zn dust (1.33 g, 20.4 mmol, 2.0 equiv.) were added. After refluxing overnight, the reaction mixture was cooled to room temperature and filtered through a pad of silica gel. The filtrate was concentrated and the crude product was purified by flash chromatography (elution: hexane/ethyl acetate=98/2). The desired compound 4 was obtained as an off white solid in 80% yield (2.93 g). Analytical data matched the published ones.

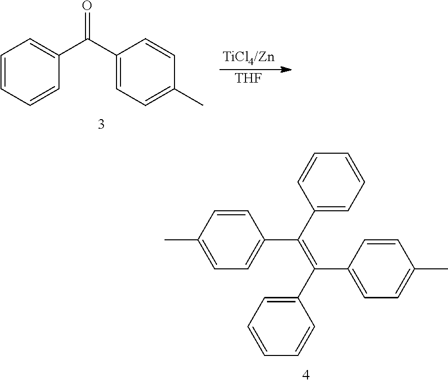

4 (1.17 g, 3.25 mmol, 1.0 equiv.), freshly recrystallized NBS (1.45 g, 8.12 mmol, 2.5 equiv.) and benzoyl peroxide (157 mg, 0.65 mmol, 0.2 equiv.) was dissolved in 108 ml of carbon tetrachloride. The solution was refluxed for 20 h. After cooled to room temperature, the solution was filtered and the filtrate was concentrated. The crude product was purified by flash chromatography (elution: hexane/DCM=6/4). The desired compound 5 was obtained as yellow oil in 50% yield (850 mg). Analytical data matched the published ones.

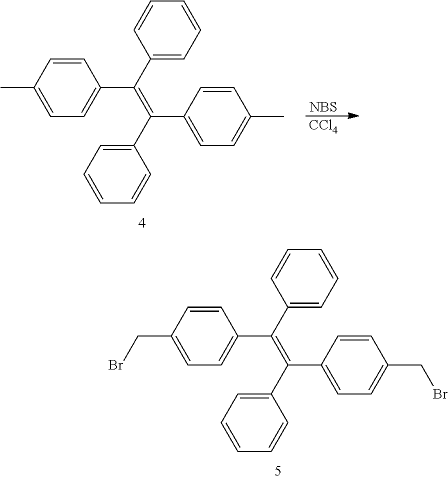

Dibromo compound 5 (850 mg, 1.64 mmol, 1.0 equiv.) and sodium azide (267 mg, 4.10 mmol, 2.5 equiv.) were dissolved in 41 ml of DMSO. After stirring at room temperature for 2 days, small amounts of water were added to quench the reaction, during which the solution temperature was increased slightly. After the solution cooled to room temperature, the solution was extracted with diethyl ether five times. The organic phases were combined, washed with water and brine, and then dried over MgSO₄. After filtration and solvent evaporation, the crude product was purified by flash chromatography (elution: hexane/DCM=9/1 to 7/3). The desired compound 6 was obtained as yellow oil in 84% yield (615 mg).

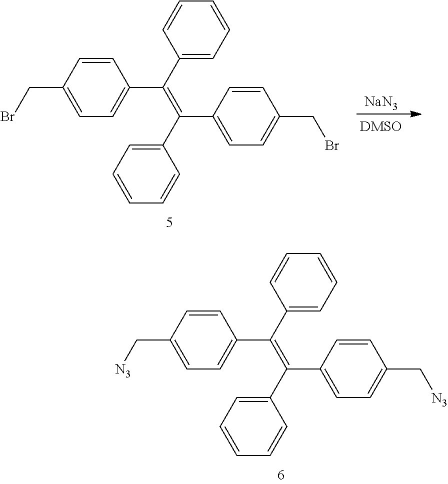 [SMILES: BrCc1ccc(/C(=C(\c2ccccc2)c2ccc(CBr)cc2)c2ccccc2)cc1 ; [N-]=[N+]=NCc1ccc(/C(=C(\c2ccccc2)c2ccc(CN=[N+]=[N-])cc2)c2ccccc2)cc1 ; "NaN3"]

Diazide compound 6 (190 mg, 0.43 mmol, 1.0 equiv.) and 4-pentynoic acid (84 mg, 0.86 mmol, 2.0 equiv.) were dissolved in 35.8 ml of t-BuOH and 7.2 ml of H₂O. Freshly prepared aqueous solutions of CuSO₄ (86 fll, c=1 M, 0.086 mmol, 0.2 equiv.) and sodium ascorbate (86 fll, c=1 M, 0.086 mmol, 0.2 equiv.) were added every few hours, until a total amount of 1 equiv. was reached for both of them. After stirring at room temperature for a total of 24 h, the starting material was still detected in the TLC. The reaction was nevertheless quenched with brine to avoid a conversion to the bi-click product only. The solution was extracted diethyl ether five times; the organic phases were combined, washed with brine, and then dried over MgSO₄. After filtration and solvent evaporation, the crude product was purified by flash chromatography (elution: DCM/MeOH=95/5 to 8/2). Compound 7 was obtained as yellow oil in 28% yield (66 mg), along with the bi-click product 8 as yellow solid in 10% yield (28 mg), and some starting material (41 mg, 21%).

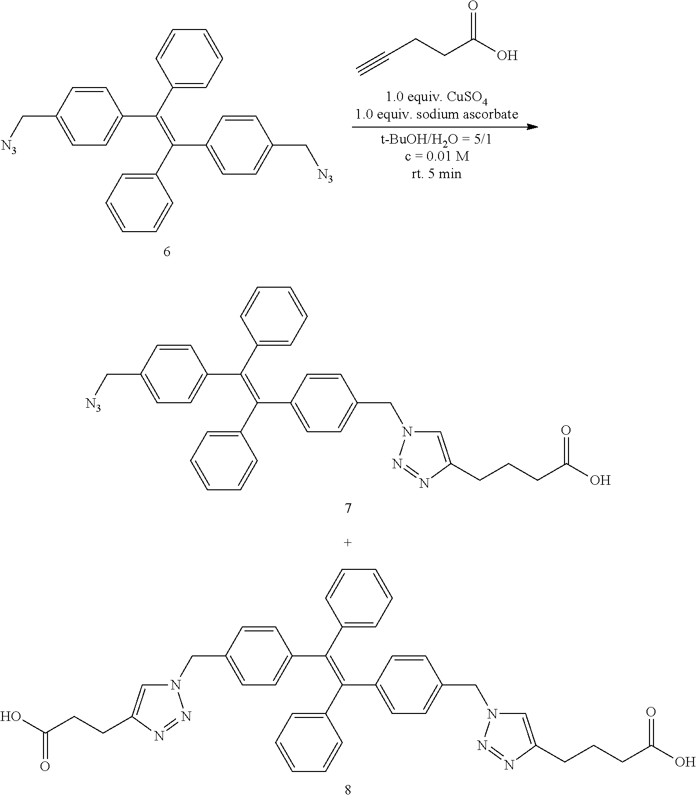

### Analysis of 7:

1H-NMR (400 MHz, CDCl₃): δ (ppm)=7.21-6.96 (m, 19H, Harom and Htriazole), 5.37 (s, 2H, Ph-CH₂-Ntriazole), 4.32 (s, 2H, Ph-CH2-N3), 3.00 (m, 2H, CH2), 2.75 (m, 2H, CH2)

13C-NMR (400 MHz, CDCl3): δ (ppm)=177.3 (C4, COOH), 146.6 (C4, Carom), 144.3 (C4, Ctriazole), 143.7 (C4, Carom), 143.2 (C4, Carom), 141.2 (C4, Carom), 140.7 (C4, Carom), 133.6 (C4, C═C), 132.8 (C4, C═C), 132.0 (C3, Carom), 131.8 (C3, Carom), 131.3 (C3, Carom), 128.0 (C3, Carom), 127.8 (C3, Carom), 127.5 (C3, Carom), 126.8 (C3, arom), 121.4 (C3, Ctriazol), 54.6 (C2, Ph-CH2-N3), 54.0 (C2, Ph-CH2-triazole), 20.9 (C2, CH2-COOH)

IR (KBr): ν (cm−1)=3414 (COOH), 2923, 2096 (N3), 1690 (COOH), 1443, 1261, 1205, 1062, 797, 697

In general, the synthesis of fluorophores containing two linkers for conjugation to a microbead and a ligand can be performed with other classes of fluorophores such as: arylenevinylene systems (e.g. cis 2,5-diphenyl-1,4-distyrylbenzene J Phys Chem B. 2006 Oct. 26; 110(42):20993-1000, DOI:10.1021/jp064069q) triphenylamine-based diketo-pyrrolo-pyrroles (Australian Journal of Chemistry 65(4) 387-394, http://dx.doi.org/10.1071/CH11410) Cholesteryl substituted pyrane derivatives (J. Phys. Chem. B, 2007, 111 (8), pp 2000-2007/DOI:10.1021/jp067374k) 8,8a-dihydrocyclopenta[a]indene-derivatives (Angewandte Chemie International Edition, 2008, 47, 51, 9891-9894, DOI:10.1002/anie.200802560) Salicylaldehyde Azines (e.g. N,N′-disalicylalacine: J. Org. Chem., 2009, 74 (5), pp 2163-2166, DOI:10.1021/jo802631m) p-phenyleneethylnylene substituted o-carboranes (Macromolecules, 2009, 42 (5), pp 1418-1420/DOI:10.1021/ma8027358) poly-triazole polymers (Macromolecules, 2009, 42 (5), pp 1421-1424, DOI:10.1021/ma8024706), polyphenylenes polyarylenes triphenylamine-fluorenones triphenylamine-benzaldehydes dendritic phosphole-sulphides diphenylfumaronitrile derivatives benzene-1,3,5-tricarboxamide derivatives poly(1-phenyl-1-alkyne)s poly(diphenylacetylene)s, 11,11,12,12,-tetracyano-9,10-anthraquinodimethane, tetraphenylethene derivative, 2,3,4,5-tetraphenylcyclopenta-2,4-dienyl benzene derivatives, compounds containing triphenylamine-anthrylenevinylene and tetraphenylethene moieties or tetraphenylthiophene moiety, 4,4′-bis(1,2,2-triphenylvinyl)biphenyl, triarylcyclopentadiene compounds, dendritic phosphole oxides, 1,8-naphthalimide based compounds, trifluoromethyl substituted distyrylbenzenes, naphthalimide derivatives, diphenyldibenzofulvene, poly(pyridinium salt)s, tetranaphthylethene compounds, benzobis(thiadiazole)-based compounds.

### Applications:

Advantageously, the disclosed method provides a generic method for binding assays on a microarray format.

More advantageously, the disclosed method may allow the formation of large libraries of microparticle assemblies carrying a large variety of ligands for pharmaceutical screening applications.

Hence, the disclosed method may overcome problems associated with the prior art, such as changes in analyte binding affinity and characteristics due to labels, multi step assay protocols, non-compatibility of different assays, limits in multiplexing, limitations in kinetic analysis and long assay times.

Advantageously, the disclosed method provides an improved method to perform bioassays, in particular multiplex bioassays on a planar bead microarray format.

Advantageously, real time measurement of kinetic binding or reaction events between a target and the ligand can be obtained. In kinetic measurements performed using the microparticle assembly, a change in optical signature over time is recorded. Advantageously, this makes the measurement independent from any initial signal or background or unspecific signal.

The disclosed method may also be combined with state-of-the-art microarray methods to improve the performance of such known methods substantially.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1. A bead construct, comprising:
(a) a bead;
(b) a fluorophore molecule, wherein the fluorophore molecule comprises at least two subunits connected to each other by at least one internal linker that provides conformational freedom to the at least two subunits, and wherein an optical signal emitted by the fluorophore molecule changes when reducing the conformational freedom of the at least two subunits to each other;
(c) a first linker molecule connected to the bead and to one of the subunits of the flurophore molecule;
(d) a second linker molecule connected to another one of the subunits of the fluorophore molecule; and
(e) a ligand molecule connected to the second linker molecule.

2. The bead construct according to claim 1, wherein the at least two subunits of the flurophore molecule is no more than two subunits.

3-7. (canceled)

8. The bead construct according to claim 1, wherein the fluorophore molecule is selected from the group consisting of: chemo- or bioluminescent molecules, aggregation-quenched fluorophores, aggregation induced fluorescent fluorophores, black-hole quenchers paired with regular fluorophores, fluorescent proteins, FRET dye pairs, metal-enhanced fluorophores, stacking-induced emission or quenching fluorophores, and Dexter and Exciplex quenchers.

9. The bead construct according to claim 8, wherein the fluorescent protein is selected from the group consisting of: GFP, RFP, YFP, CFP and constructs thereof.

10. The bead construct according to claim 8, wherein the FRET dye pairs are selected from the group consisting of: mOrange2/mCherry, CFP/YFP and FITC/TRITC.

11. The bead construct according to claim 8, wherein the chemo or bioluminescent molecule is selected from the group consisting of: Rhodamine, Fluorescein, Coumarin, Acridin, Bodipy, Dansyl, Dapoxy, Hoechst, Cy, merocyanines, DAPI, SYTO, Alexa Fluor dyes, Pechmann dyes, polymethines, biaryl or bi(heteroaryl) dyes, thiophenes, helicines, phenylenes, xanthenes, naphthylenes and their derivatives, isomers and conjugates thereof, photochromic molecules and aggregation-induced emitters.

12. The bead construct according to claim 11, wherein the photochromic molecules are selected from the group consisting of: spiropyrans, spirooxazines, quinones, diaryethenes, azobenzenes, and their derivatives, isomers and conjugates thereof.

13. The bead construct according to claim 11, wherein the aggregation-induced emitters are selected from the group consisting of: tetraphenylethene, hexaphenylsilole, diphenyldibenzofulvene, siloles and their derivatives, isomers and conjugates thereof.

14. The bead construct according to claim 8, wherein the metal-enhanced fluorophores are selected from the group consisting of: metal-ligand complexes based on porphyrins, bipyridyl and phenanthrolines.

15. The bead construct according to claim 8, wherein the stacking-induced emission or quenching fluorophores are selected from the group consisting of: perylene, pyrene, terylene and their derivatives, isomers and conjugates thereof.

16. The bead construct according to claim 8, wherein the fluorophore molecule is an aggregation-induced fluorophore, or a mono-component, or a di-component fluorophore.

17. The bead construct according to claim 1, wherein the bead has a diameter from 50 nm to 100 micrometer.

18. The bead construct according to claim 1, wherein the first linker molecule has a length in a range of 0.2 nm to 500 nm.

19. The bead construct according to claim 1, wherein the first linker molecule comprises an organic polymer.

20. The bead construct according to claim 19, wherein the organic polymer is selected from the group consisting of: polyethylene glycol, poly(vinylpyrrolidone), oligo(ethylene glycol), polyacrylamide, dextran, poly-2-methyl-2-oxazoline, poly(carboxybetaine methacrylate), poly(L-lysine), poly(styrenesulfonate), polyanhydride, poly(acrylic acid), polyethyleneimine, polyglycolic acid and polycaprolactone.

21. The bead construct according to claim 1, wherein the second linker molecule has a length in a range of 0.1 nm to 10 nm.

22. The bead construct according to claim 1, wherein at least one of the first and the second linker molecule is selected from the group consisting of: 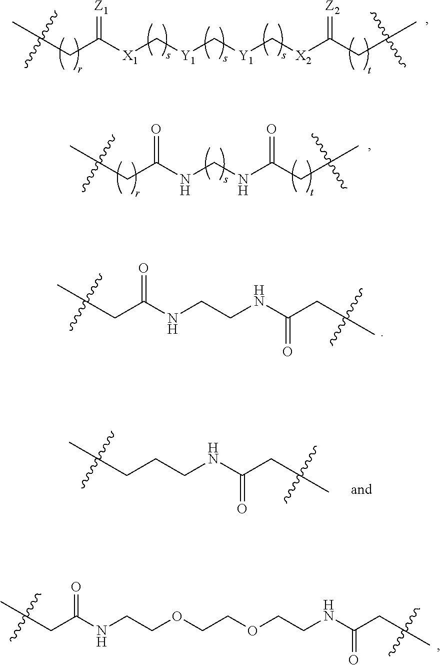
wherein
each of r, s, and t are independently selected from 0, 1, 2, or 3;
w is 1;
X₁, X₂, Y₁ and Y2 are independently selected from CR₂, O, S, or NR, wherein R is selected from the group consisting of an alkyl group, an acyl group, an aryl group, a cycloalkyl group and a heterocycle group, or R forms an aryl group, a cycloalkyl group or a heterocycle group with another carbon atom within the linker; and
Z₁ and Z₂ is independently selected from O, S or H₂.

23. The bead construct according to claim 1, wherein the ligand is selected from the group consisting of: a drug, a metabolite, a peptide, an antigen, a biotin, a hormone, a steroid, a toxin, an antibiotic, an allergen, a protein, a cofactor, a metal, pesticide, insecticide, oligonucleotide, polymer, biopolymer, saccharide, lipid, an electrochemically reactive ligand, parts thereof, derivatives thereof and mixtures thereof.

24. A microarray, comprising a plurality of bead constructs disposed thereon, wherein the bead constructs comprise:
(a) a bead;
(b) a fluorophore molecule, wherein the fluorophore molecule comprises at least two subunits connected to each other by at least one internal linker that provides conformational freedom to the at least two subunits, and wherein an optical signal emitted by the flurophore molecule changes when reducing the conformational freedom of the at least two subunits to each other;
(c) a first linker molecule connected to the bead and to one of the subunits of the flurophore molecule;
(d) a second linker molecule connected to another one of the subunits of the fluorophore molecule; and
(e) a ligand molecule connected to the second linker molecule.

25. The microarray according to claim 24, wherein the plurality of bead constructs allows identification of a presence of a plurality of target analytes in a sample.

26-31. (canceled)

32. A kit for identifying the presence of a target analyte in a sample, the kit comprising:
a microarray according to claim 24, one or more reagents and instructions for use.

33. The kit according to claim 32, wherein the one or more reagents are selected from the group consisting of: a fluorescent labeled binding molecule, an antibody, a receptor and an aptamer.
